(19) United States
(12) Patent Application Publication
(10) Pub. No.: US 2022/0022568 A1
(43) Pub. Date: Jan. 27, 2022

(54) P.A.C.K (Portable Air Conditioner Kit)

(71) Applicant: Gregory Kamau Jackson, Snellville, GA (US)

(72) Inventor: Gregory Kamau Jackson, Snellville, GA (US)

(21) Appl. No.: 16/947,177

(22) Filed: Jul. 21, 2020

(51) Int. Cl. A41D 13/005; A61F 7/00

(52) U.S. Cl. CPC A41D 13/0058; A61F 2007/0023; A61F 7/00

(57) ABSTRACT

The P.A.C.K is designed to provide the individual with continuous refreshing air during extreme weather. The P.A.C.K mounts a fan pointed upward towards the users' body. The innovative aspect of the P.A.C.K is that it is a “hands free” portable device that allows the user to be active all the while with both hands available for work unlike the existing devices that requires the use of one hand limiting the activity of the user. This is achieved by attaching the unit to the users' pant belt. The P.A.C.K has an element guard that channels the air towards the body.

### BACKGROUND OF THE INVENTION

The P.A.C.K attempts to solve the awkward and inconvenient method of cooling the human body during extreme hot weather. Currently in order to cool the body one will have to stand in front of a stationary cooling source to feel comfortable. In addition, one has the option of a portable fan, but one would have to tote it using one hand limiting their activity. The P.A.C.K solves all mentioned problems by making the cooling source portable all the while freeing both hands ultimately making the individual comfortable and fully functional.

### BRIEF SUMMARY OF THE INVENTION

The P.A.C.K is a hands free device that solves the problem of losing a whole arm in the attempts of cooling the body while on the go. The P.A.C.K achieves this by attaching a cooling element to the users' belt making the user feel comfortable and fully active.

The P.A.C.K (Personal Air Conditioning Kit)

### BRIEF DESCRIPTION OF THE SEVERAL VIEWS OF THE DRAWINGS

FIG. 1: Shows the Front View of The P.A.C.K

FIG. 2: Shows the Left Side View of The P.A.C.K

FIG. 3: Shows the Isometric View of The P.A.C.K

FIG. 4: Shows the Electrical Schematic of The P.A.C.K

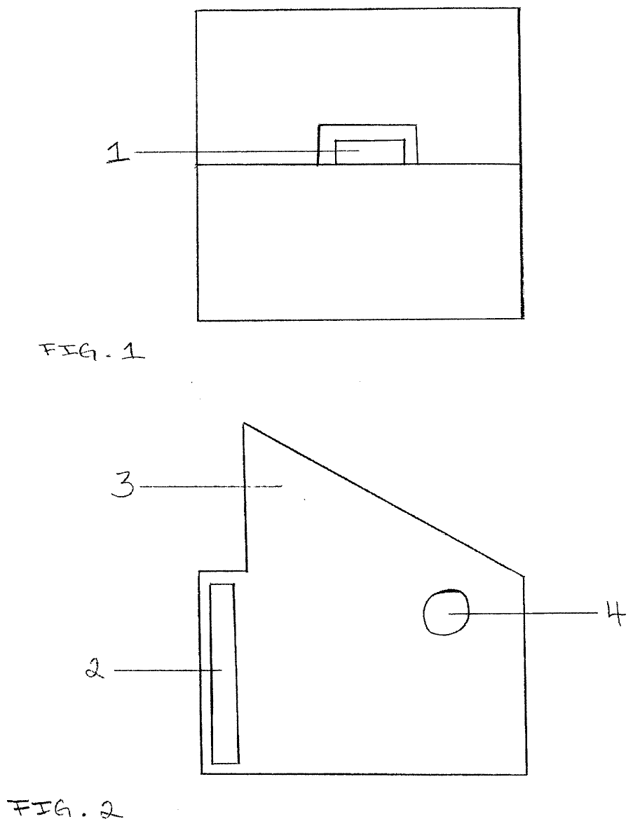

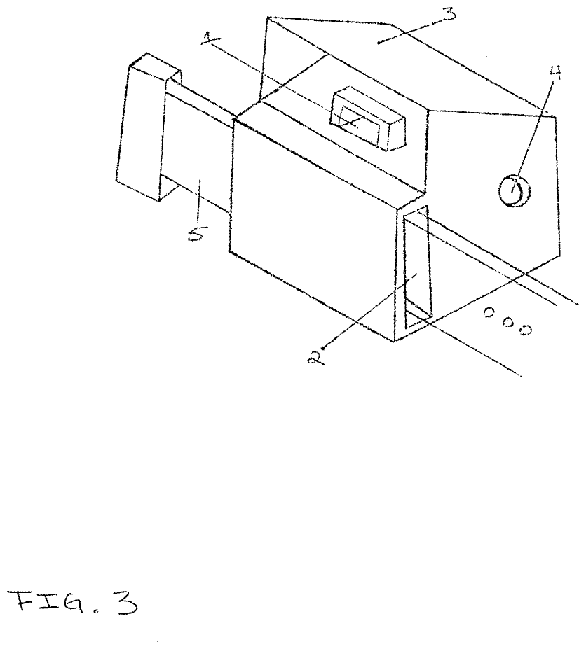

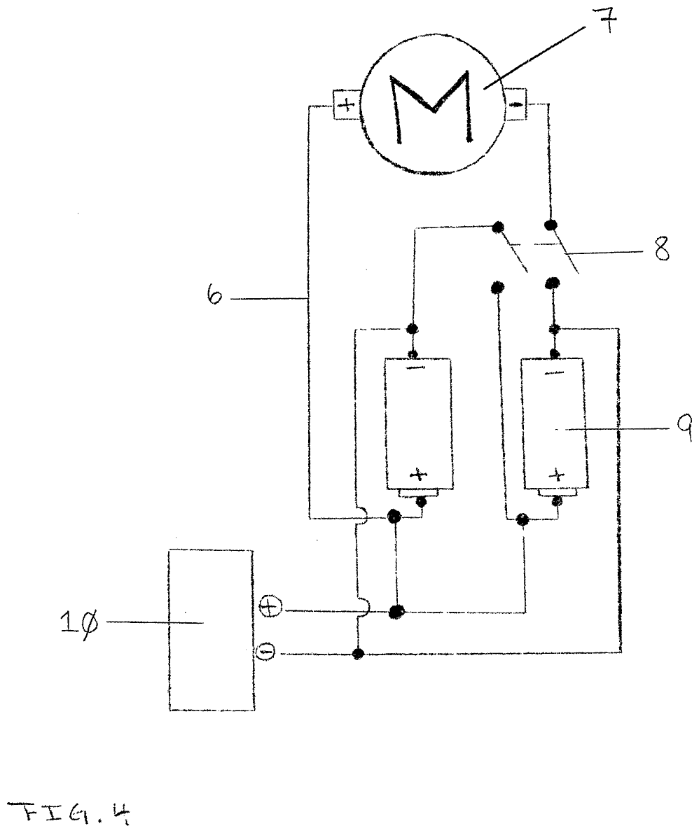

The P.A.C.K is a plastic compact shaped housing with an (1) element mount that holds a cooling element which will be a fan driven by a (7) motor aimed upward. The P.A.C.K is (8) switch controlled connected to (9) batteries in series configuration. The batteries are charged up after depletion by a (10) battery charger. All components mentioned ((8) switch; (7) motor; (9) batteries; (10) battery charger) are connected by (6) copper wire. The (8) switch is routed through the (4) switch hole. The P.A.C.K has a (2) belt loop on the front of the housing to harness a (5) belt.

The manner and process of making the P.A.C.K will be by way of plastic injection molding and the electronic devices will be purchased by electronic suppliers.

The P.A.C.K will be utilized by placing the users' (5) belt through the (2) belt loop of the P.A.C.K until it reaches the center of the belt, then the users upper garment (shirt) will cover the (3) guard of the P.A.C.K so that the unit is totally underneath the upper garment with the cooling element pointed directly toward the users' bare body in turn cooling the body temperature of the user.

The P.A.C.K sets itself apart from any “hand held” personal fan because the P.A.C.K is a “HANDS FREE” device.

1. The P.A.C.K is a portable and personal hands-free “wearable” apparatus that conducts cooling air directly into the ambient environment then ultimately to the users' body. The air is provided by a fan propeller driven by a motor that sits center of the apparatus on its element mount. The P.A.C.K is a plastic compact square shaped housing comprised of a flat bottom with a dome shaped shirt guard as the roof to conduct cooling air and prevents the wearers shirt from interfering with the air flow. The flat bottom of the P.A.C.K makes it a desktop fan as well as a hands-free wearable apparatus. The P.A.C.K does not use any secondary accessories to conduct cooling air, instead it uses a dome shaped shirt guard as a roof to do so. The apparatus is attached to the user by routing the users pant belt through the apparatus belt loop and underneath the users' shirt.
